(19) United States
(12) Patent Application Publication
(10) Pub. No.: US 2024/0277599 A1
(43) Pub. Date: Aug. 22, 2024

(54) Compositions That Improve Sunscreen Performance

(71) Applicant: ELC Management LLC, Melville, NY (US)

(72) Inventors: Wilson A. Lee, Hauppauge, NY (US); Monica Apostol, Coram, NY (US); Jing Qi, Deer Park, NY (US); Muriel Ghersin, Paris (FR)

(21) Appl. No.: 18/168,776

(22) Filed: Feb. 14, 2023

(51) Int. Cl. A61K 8/81; A61Q 17/04

(52) U.S. Cl. CPC A61K 8/8152; A61K 8/8135; A61Q 17/04

(57) ABSTRACT

Film forming compositions that are able to improve the effectiveness of sunscreen preparations, especially those comprising inorganic sunscreen agents ZnO or TiO₂. Compositions according to the invention comprise specific combinations of acrylates/VA copolymer and acrylates copolymer, in a cosmetically acceptable base or delivery vehicle. These copolymers may be incorporated into a composition that comprises physical sunscreen agents, or they may be incorporated into a primer composition that is applied directly to the skin, followed by a composition that comprises physical sunscreen agents.

### FIELD OF THE INVENTION

The present invention is in the field of photoprotective compositions. More specifically, the invention pertains to film forming compositions that improve the performance of sunscreen preparations.

### BACKGROUND

The sun emits a spectrum of ultraviolet radiation between about 100 nm and 400 nm wavelength. The portion of the spectrum between about 290 nm and 320 nm is known as UV-B (UVB), and that between about 320 nm and 400 nm is known as UV-A (UVA). The portion of the UVA spectrum from 340 nm to 400 nm has been further designated as UVAI (long-wave UVA). Much, but not all, of the UV-B radiation from the sun is absorbed by the Earth's ozone layer, while the UV-A radiation is not significantly absorbed in the atmosphere. Both UV-A and UV-B are known to produce harmful effects in human skin.

UVB rays are higher in energy than UVA rays, and are much more effective at producing sunburn and cellular damage, including DNA damage. UVA rays are less effective at producing sunburn than UVB rays. However, UVA makes up the majority of UV radiation, and penetrates much deeper into the skin than UVB. Thus, UVA rays have the potential to encourage formation of reactive oxygen species (ROS) which cause oxidative damage to skin pigment cells. UVA rays also contribute to photo-aging, and to the formation of DNA damage, albeit to a lesser degree than UVB.

Sunscreen products are applied to human skin to protect the skin by filtering or blocking harmful UVA and UVB rays in sunlight. The active ingredients in sunscreen products fall broadly into two categories; chemical and physical sunscreen agents. Chemical sunscreen agents are organic molecules that absorb ultraviolet radiation, and convert it into a less hazardous type of energy. Chemical sunscreen agents may be further categorized according to whether their primary effect is in the UV-A or UV-B portions of the spectrum. In the United States, the following UV-A absorbers have been historically common: avobenzone, dioxybenzone, ecamsule, meradimate, oxybenzone and sulisobenzone. Chemical UV-B absorbers have included cinoxate, ensulizole, homosalate, octinoxate, octisalate, octocrylene PABA, padimate O and trolamine salicylate. The second category of sunscreen agents are physical sunscreen agents. These are inorganic molecules, such as titanium dioxide and zinc oxide, that absorb, reflect and scatter light, changing its wavelength in the process. Titanium dioxide (TiO₂) is a moderately potent UVB absorber, and a somewhat less good UVA absorber. On the other hand, zinc oxide (ZnO) is considered effective against both UVA and UVB rays, but less so against UVB.

In recent years, the need has arisen to better understand the general safety of some sunscreen active agents. As a result, some chemical sunscreen active ingredients have fallen out of favor, and their use has been curtailed or eliminated. On the other hand, the physical sunscreen actives, titanium dioxide and zinc oxide, continue to be regarded as generally safe and effective, and these have emerged as the sunscreen actives of choice. However, there have always been downsides to physical sunscreens. These include formula stability issues. Physical sunscreen agents are relatively dense. Over time, they tend to settle out of fluid compositions, which makes their application inconsistent, and the supplied sun protection variable. In order to ensure that the level of SPF claimed on the label is made available to a consumer, manufacturers of sunscreen products tend to overload their products. This can be expensive and aesthetically unpleasant, resulting in a chalky appearance and a heavy, uncomfortable feel on the skin.

Co-owned application, U.S. Ser. No. 17/535,556, discloses a water-resistant sunscreen composition, comprising one or more sunscreen actives, and at least two film formers. Required film formers are an acrylates copolymer and a xanthan gum in a ratio from about 15:1 to about 1:1. In the following co-owned applications, U.S. Ser. No. 15/632,903 (now U.S. Pat. No. 11,103,439), U.S. Ser. No. 15/906,372 (now U.S. Pat. No. 10,813,874), U.S. Ser. No. 17/029,147, U.S. Ser. No. 17/176,527, U.S. Ser. No. 16/816,995, U.S. Ser. No. 16/197,858, U.S. Ser. No. 16/381,806 (now U.S. Pat. No. 10,507,175), U.S. Ser. No. 16/267,441 (now U.S. Pat. No. 10,980,717), U.S. Ser. No. 17/189,768, U.S. Ser. No. 16/827,876 (now U.S. Pat. No. 11,129,788), and U.S. Ser. No. 17/806,294 (all incorporated herein by reference in their entirety) the applicant has disclosed specific combinations of acrylates/VA copolymer and acrylates copolymer in an aqueous base, and various useful properties of these combinations depending on the application and the presence of certain other ingredients. Upon application to a skin surface, these compositions, although water based, dry to a film that is clear, flexible and comfortable, as well as able to resist water breakdown as long as the water temperature is below a certain minimum.

More specifically, U.S. application Ser. No. 17/806,294 discloses topical film forming compositions that are able to efficiently deliver active ingredients to keratinic surfaces in a timed release manner. The compositions comprise a film former that comprises acrylates/VA copolymer and acrylates copolymer in a weight ratio of 10:1 to 30:1, and a combination of propane-1,3 diol and glycerin. The weight of the acrylates/VA copolymer and acrylates copolymer combined comprise 20%-60% of the weight of the total composition. It is reported that the sizes of the pores that develop in the dried film may be controlled or fine tuned.

U.S. Pat. No. 10,980,717 discloses reduced-ethanol perfume compositions that preserve aromatic integrity, the compositions comprising 4.5% to 18.5% by weight of acrylates/VA copolymer, 0.25% to 1.0% by weight of acrylates copolymer in an aqueous base, and one or more materials in the aqueous phase that are able to plasticize the acrylates/VA copolymer, and modify the porosity of the resulting film upon drying. It is reported that, in general, more plasticizer in the aqueous phase tends to increase the size of the surface pores that develop in the acrylates/VA copolymer-acrylates copolymer film. Controlling this pore size is key to controlling the release of fragrance as a function of time, and maintaining the integrity of the perfuming ingredients in the composition. Particularly good results were reported when the weight ratio of acrylates/VA copolymer to acrylates copolymer is in the range of 10:1 to 30:1

None of the foregoing references discloses film forming compositions that are able to improve the effectiveness of sunscreen preparations, especially those comprising inorganic sunscreen agents ZnO or TiO₂. There remains a need for improved photoprotective sunscreen compositions.

### SUMMARY

Compositions according to the present invention comprise specific combinations of acrylates/VA copolymer and acrylates copolymer, in a cosmetically acceptable base or delivery vehicle. These copolymers may be incorporated into a composition that comprises physical sunscreen agents, such as titanium dioxide and zinc oxide. Or, they may be incorporated into a primer composition that is applied directly to the skin, followed by a composition that comprises physical sunscreen agents.

Following application to the skin, the acrylates/VA copolymer and acrylates copolymer dry to a film in the form of a multi-layer network of pores that have a preferred size, and preferred Young's modulus. When employed as herein described, compositions of the present invention significantly boost the SPF in the critical range of about 200 nm to about 420 nm. Compositions according to the invention may or may not be sprayable with a conventional cosmetics pump sprayer.

### DETAILED DESCRIPTION

Except in operating and comparative examples, or where otherwise explicitly indicated, all numbers in this description indicating amounts or ratios of material or conditions of reaction, physical properties of materials and/or use are to be understood as modified by the word “about.” All amounts are presented as percentages by weight of the final composition, unless otherwise specified.

Throughout the present specification, “film former” or the like refers to a polymer that leaves a film on the substrate to which it is applied, for example, after a solvent accompanying the film former has evaporated, absorbed into and/or dissipated on the substrate.

“Comprising” and the like, mean that a list of elements may not be limited to those explicitly recited.

“Sunscreen products” contain any component able to absorb, reflect or scatter UV rays, the products are intended to be placed on the surface of human skin with the purpose of protecting against UV-induced erythema (sunburn) and other ultraviolet induced skin damage. There are two main classes of sunscreen active ingredients: organic and inorganic.

“Minimal Erythema Dose” (MED) is the smallest UV dose that produces perceptible redness of the skin (erythema) with clearly defined borders at 16 to 24 hours after UV exposure.

“Sun Protection Factor” (SPF) is a relative measure of a sunscreen product's ability to protect skin from UV-induced erythema (sunburn). The SPF test measures the amount of UV radiation exposure it takes to cause sunburn when a person is using a sunscreen compared with how much UV exposure it takes to cause sunburn when the person is not using a sunscreen. SPF is related to MED in that the SPF of a sunscreen product when applied to skin increases the individual's MED.

“Critical wavelength” (CW) is the wavelength below which 90% of the total solar UV absorbance of the sunscreen product resides. The higher the CW the greater the breadth of protection against the entire UV spectrum, 290 nm to 400 nm.

A spectrophotometer is an instrument that measures the light transmission properties of a material as a function of wavelength.

### Acrylates/VA Copolymer

A first main ingredient of the invention is the acrylates/VA copolymer whose monomers are ethenyl acetate and 2-ethylhexyl prop-2-enoate; CAS number 25067-02-1, C₁₄H₂₂O₆. For detailed information, see PubChem Compound Database; CID=168269.

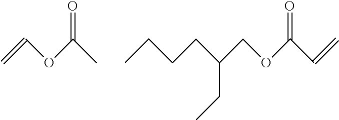

In cosmetics, this tacky material often functions as a binder, film former, adhesive and/or hair fixative. When deployed in aqueous cosmetic systems above-identified acrylates/VA copolymer can impart a film on the skin or hair. The pure acrylates/VA copolymer film features a temperature dependence, such that a water rinse of about 38° C. or more will degrade the film, and allow it to be removed from a surface, while retaining its integrity at temperatures at or below normal skin temperature (i.e. 36.5-37.5° C.).

Compositions according to some preferred embodiments of the present invention comprise about 5% to 30% of acrylates/VA copolymer by total weight of the composition, for example 10% to 20%, preferably about 10% by total weight of the composition.

The above-identified acrylates/VA copolymer is commercially available, for example, as Vinysol 2140L from Daido Chemical Corp. Vinysol 2140L is a 46.6% aqueous mixture of acrylates/VA copolymer. Therefore, when using Vinysol 2140L, in order to achieve the concentrations of acrylates/VA copolymer noted above, the concentration of Vinysol 2140L should be about 10.7% to 64.4%, for example 21.5% to 42.9%, preferably about 21.5% by total weight of the composition. Vinysol 2140L is reported to have a pH of 4.5, a viscosity of 2,000 mPa-s, a calculated glass transition temperature (Tg) of −9° C., while the film exhibits a break elongation of 1,200%, and a break strength of 1.2 MPa (when spread to a thickness 0.1 mm). By itself, the acrylates/VA copolymer is somewhat too rigid for consumer acceptance.

### Acrylates Copolymer

To address the problem of high rigidity, the acrylates/VA copolymer was combined with an acrylates polymer that has a lower Tg than acrylates/VA copolymer. In general, a lower Tg provides more flexibility to the resulting film. It also increases the dry time of the film to a useful degree. Of course, a film forming composition that dries too fast or too slow is not commercially viable. In the present invention, a suitable dry time and the right amount of flexibility in the dried film are provided by a second main ingredient: the acrylates copolymer whose monomers are ethyl prop-2-enoate; methyl 2-methylprop-2-enoate, 2-methylprop-2-enoic acid; CAS number 25133-97-5 (C₁₄H₂₂O₆).

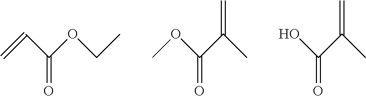

For detailed information, see PubChem Compound Database; CID=168299. In various types of cosmetic formulations, acrylates copolymers have a wide variety of uses including as film formers, hair fixatives, binders, and suspending agents, viscosity enhancers, antistatic agents and adhesives.

In the present invention, useful concentrations of acrylates copolymer are from 0.25% to 1.5%, for example 0.50% to 1.0%, preferably about 0.5% based on total weight of the composition. The above-identified acrylates copolymer is commercially available, for example, as Daitosol 5000AD from Daito Kasei Kogyo Co. Daitosol 5000AD is a 50% aqueous mixture of acrylates copolymer. Therefore, in order to achieve the concentrations of acrylates copolymer noted above, the concentration of Daitosol 5000AD should be about 0.5% to 3%, for example 1.0% to 2.0%, preferably about 1.0% by total weight of the composition. Daitosol 5000AD is reported to have a pH of 5.5-7.5, a viscosity of 50-100 mPa-s, a glass transition temperature (Tg) of about −14° C. Based on this, we can say that the ratio of the weight of acrylates/VA copolymer to the weight of acrylates copolymer is in the range 3.3:1 to 120:1. Preferably this ratio is 10:1 to 40:1, more preferably 10:1 to 30:1, even more preferably 18:1 to 20:1.

### Physical Sunscreens

Some preferred embodiments of the invention comprise one or more physical sunscreens, such as TiO₂ and ZnO. Some preferred embodiments of the present invention comprise about 1% to about 20% of all physical sunscreens, based on total weight of the composition.

### Water

Compositions of the invention are aqueous, and may typically comprise about 20% to about 70% of water by weight of the total composition. This amount of water is that from all sources, such as that in Vinysol 2140L and Daitosol 5000AD.

### Pore Matrix

As noted above, after drying on a substrate, compositions of the invention will set up as a film that comprises a multi-layer matrix of pores. We have noted that compositions wherein the average pore size is about 1 μm to 5 μm (for example, 1.5 μm to 3 μm) are useful to improve sunscreen performance. Furthermore, individual pores as small as 0.3 μm (300 nm) have been observed. Thus, the sizes of the smaller pores in the matrix overlaps the UVA wavelength range (320-400 nm) and a portion of the UVB wavelength range (290-320 nm), and this may account for at least some of the ability of compositions of the invention to provide improved protection against ultraviolet light. Furthermore, as the composition dries, physical sunscreen particles collect in the pores of the film, which makes the distribution of TiO₂ and/or ZnO multi-level and regular. This is an improvement over the conventional physical sunscreen compositions wherein the relatively dense sunscreen agents tend to settle in the composition, resulting in inconsistent sun protection. As an added benefit, because the physical sunscreen particles (i.e. (TiO₂ and/or ZnO) collect in the pores of the dried film, there is less opportunity for the sunscreen particles to penetrate the skin.

### Plasticizer

Compositions of the invention may include one or more plasticizers in the specified amounts. These materials offer several benefits, such as increased sprayability of the wet composition. Without the plasticizer, it would be difficult, if not impossible, to spray the composition from a mechanical pump sprayer of the type commonly used in the cosmetic industry. At best, a narrow stream of product is produced, with little or no atomization upon striking the atmosphere. This is unacceptable for a product that is intended to cover a relatively large area with a thin film by spraying.

As another benefit, plasticizer may also be used to adjust the Young's modulus of the film that dries on the skin. Young's modulus is a measure of the stiffness of a material under tension or compression. A lower Young's modulus implies a less stiff material. The Young's modulus of human skin typically varies between about 0.42Mpa-0.85 MPa. A film that has formed on the skin, feels more comfortable when it's Young's modulus is lower than that of the skin (that is, when the film is more flexible than the skin). Preferred compositions of the invention produce a film that has a Young's modulus that is lower than that of the skin. More preferred compositions of the invention produce a film that has a Young's modulus that is less than half the Young's modulus of the skin. Even more preferred is when the Young's modulus of the film is less than one third the Young's modulus of the skin. And these are achievable with the present invention.

Plasticizer may also be useful, to some degree, to adjust the pore sizes in the film that dries on the skin, and fine tune the UV protection benefit. In general, more plasticizer tends to increase pore size, so its use should be limited so as not to lose too much of the improvement in sun protection. For these reasons, some embodiments of the present invention comprise one or more of butylene glycol, propanediol and glycerine. Preferred compositions of the invention comprise 1% to 5% based on total weight of the composition of butylene glycol, propanediol, glycerine, or any combination thereof.

### Surfactants and Emulsifiers

One or more surfactants or emulsifiers may also be used to adjust surface tension. As noted above, compositions of the invention typically comprise from about 0.5% to about 2% of water by weight of the total composition. Some preferred embodiments of the present invention are single aqueous phase compositions, and have little to no oil or silicone. In other preferred embodiments the compositions are lightly emulsified oil-in-water emulsions. The emulsion embodiments are useful when a composition comprises fragrance oils, or when the composition will be used to deliver at least one oil soluble actives (such as vitamin E acetate) to a keratinic surface. However, one or more surfactants or emulsifiers can also be used in the present invention to reduce the surface tension and increase sprayability of spray-on film forming compositions. In general, increasing the level of surfactant or emulsifier will lower the surface tension of the film forming compositions. Whether used to adjust surface tension or to emulsify oil soluble ingredients, the one or more surfactants or emulsifiers should have an HLB between 8 and 12, and comprise no more than 2% of the total composition, typically between 0.01% to 2% of the total composition.

### Hydrophobic Materials

Prior to applying to a keratinic surface, the film forming compositions of the present invention are in a first or hydrophilic state. The ability to formulate with water soluble ingredients in this first state is advantageous. To maintain sufficient hydrophilicity in the first state, the use of hydrophobic materials should be limited to less than about 5% based on total weight of the composition, for example 0.001% to 5%; preferably less than 2%, more preferably less than about 0.25%. Materials that are partly hydrophilic and partly hydrophobic could possibly exceed these limits, based on the performance of the final composition. In some embodiments of the invention, it is preferable if the composition comprises no hydrophobic ingredients, such as hydrophobic oils or waxes. Oils are organic substances that are liquid at ambient temperature, such as esters, triglycerides, hydrocarbons and silicones. A typical wax used in cosmetic compositions is carnauba wax. In some embodiments of the invention, it is most preferable if the compositions contain no hydrophobic oils or waxes.

### Polyurethane

Polyurethane tends to make compositions very rigid, and inhibits pore formation. Therefore, film forming compositions of the invention comprise no more than 0.5%, for example 0.0001% to 0.5%, of polyurethane. More preferably, compositions of the invention comprise no polyurethane.

### Various Ingredients

Various ingredients may be included in the film forming compositions to fine tune the consumer experience or enhance the performance of the composition and the adjacent cosmetic or skincare preparation. Alcohols, for example, may be useful to speed up drying after application to the skin. Amounts of alcohol up to 5% may be useful. The film forming compositions may also comprise preservatives and antioxidants, typically up to about 2% by weight of the composition. Thickeners, viscosity decreasing agents, and/or pH adjusters (such as caustic soda) may be used as needed to create a consumer acceptable product, typically at levels of less than 1% by weight of the composition. At these levels, the foregoing named ingredients do not seem to adversely affect the useful properties of the film forming composition.

Compositions of the invention may or may not comprise pigments. When pigment is present, preferred compositions will comprise a total of no more than 1%, for example 0.001% to 1% of pigments.

Active ingredients may be incorporated into the aqueous phase or oil phase (if there is one). Examples of hydrophilic (water soluble) actives include: algae extract, Alpinia speciosa leaf extract, Alteromonas ferment extract, ascorbyl acid glucoside (AA2G), Citrullus lanatus (watermelon) fruit extract, Crataegus monogyna (hawthorn) flower extract, hyaluronic acid, hydrolyzed yeast protein, Lactobacillus ferment, Matricaria (chamomile) extract, lens Esculenta (lentil) fruit extract, Paeonia suffruticosa (peony) root extract, panthenol, Pyrus malus (apple) fruit extract and Saccharum officinarum extract. Each individual hydrophilic active is typically incorporated at no more than 5.0%, for example 0.0001% to 5%, by weight of the composition. Examples of hydrophobic (oil soluble) actives include Anthemis nobilis oil, bht (butylated hydroxytoluene), caffeine, Cocos nucifera (coconut) oil, salicylic acid, tetrahexyldecyl ascorbate and tocopheryl acetate. Each individual hydrophobic active is typically incorporated at no more than 1%, for example 0.0001% to 1%, by weight of the composition.

### Absorption Analysis 1

Compositions according to the present invention comprise specific combinations of acrylates/VA copolymer, acrylates copolymer and a physical sunscreen, in a cosmetically acceptable aqueous base. Upon drying, the acrylates/VA copolymer and acrylates copolymer form a film that comprises a multi-layer matrix of pores. The ability of this film to significantly boost SPF in the range of about 200 nm to about 420 nm, may first be demonstrated by determining how much light is absorbed by the matrix. A 20:1 mixture of acrylates/VA copolymer and acrylates copolymer was spread on a PMMA petri dish and allowed to dry to a thin film, approximately 1.5 mm thick. A quartz halogen lamp (Britek Halo Flood 800/650PS) at a distance of 12 inches served as a source of visible and UVA radiation. A UVP® 95-0313-01 model: MRL-58 with a UVB bulb at a distance of 4 inches served as a source of UVB radiation. Absorbance was measured using Solar Light model: PMA2100 dual-input datalogging radiometer, with UVA detector PMA2110, UVB detector PMA2106, and Photopic detector PMA2130.

Measurements were made of light intensity (W/m²) as follows: light passing through air only; light passing through air and a clean PMMA petri dish; light passing through air, dried film test sample and PMMA dish.

| | | | % Decrease |
|---|---|---|---|
| | | | in intensity |
| | Air + | Air + film + | at detector |
| | PMMA dish | PMMA dish | (Absorbance |
| Radiation | (W/m²) | (W/m²) | by film) |
| UV-B | 8.22 ± 0.06 | 0.004 ± 0.005 | 99.95 ± 0.06% |
| UV-A | 2.00 ± 0.02 | 1.05 ± 0.02 | 47.64 ± 1.38% |
| Visible | 65.87 ± 0.36 | 53.86 ± 0.10 | 18.23 ± 0.33% |

The results clearly indicate that the dried polymer film is much better at filtering UV-A and UV-B radiation than visible light. Thus, the polymer film is selective for UV-A and UV-B absorbance. We suspect this selectivity has something to do with the size of the pores in the polymer matrix.

### Absorption Analysis 2

We next measured the absorbance of UVA and UVB radiation by a full formula physical sunscreen product that incorporates the acrylates/VA copolymer and acrylates copolymer combination. The analysis utilized two control samples and one test sample. A base formula comprising 10% zinc oxide served as control sample 1. Control sample 1 may be considered as a conventional physical sunscreen product. The test sample was constituted by the base formula (conventional sunscreen product) plus the two polymers, acrylates/VA copolymer and acrylates copolymer, in a 18.6:1 ratio. Control sample 2 was constituted by the base formula with zinc oxide removed, and the two polymers, acrylates/VA copolymer and acrylates copolymer, added in a 18.6:1 ratio. The test and control samples are shown in the following table.

| | Test | Control | Control |
|---|---|---|---|
| Ingredient | Sample | Sample 1 | Sample 2 |
| Water | q.s. | q.s. | q.s. |
| 1,3 butylene glycol | 0.76 | 0.76 | 0.76 |
| PEG-10 Dimethicone | 0.57 | 0.57 | 0.57 |
| glycerine | 0.38 | 0.38 | 0.38 |
| dimethicone | 0.38 | 0.38 | 0.38 |
| dimethicone, | 0.28 | 0.28 | 0.28 |
| polysilicone-11 | | | |
| polyethylene glycol | 0.19 | 0.19 | 0.19 |
| cyclotetrasiloxane/ | 0.10 | 0.10 | 0.10 |
| isododecane/ | | | |
| polysilicone-11 | | | |
| isododecane | 0.10 | 0.10 | 0.10 |
| ¹ Aristoflex AVC | 0.08 | 0.08 | 0.08 |
| phenoxyethanol | 0.57 | 0.57 | 0.57 |
| ² Barguard ® | 0.06 | 0.06 | 0.06 |
| CP/Jeecide ™ CAP-2 | | | |
| Vitamin E | 0.04 | 0.04 | 0.04 |
| ³ Carbopol ® ultrez 30 | 0.02 | 0.02 | 0.02 |
| polymer | | | |
| hyaluronic acid | 0.02 | 0.02 | 0.02 |
| caustic soda 30% | 0.02 | 0.02 | 0.02 |
| EDETA BD/disodium | 0.01 | 0.01 | 0.01 |
| EDTA | | | |
| cellulose nanofiber | 4.50 | 4.50 | 4.50 |
| zinc oxide | 10.0 | 10.0 | |
| acrylates/VA copolymer | 9.32 | | 9.32 |
| acrylates copolymer | 0.50 | | 0.50 |

¹ Ammonium Acryloyldimethyltaurate/VP Copolymer
² Caprylyl Glycol/Phenoxyethanol/Hexylene Glycol
³ cross-linked homopolymer of polyacrylic acid (thickener)

### Procedure

The test is based on the assessment of UV-transmittance through a thin film of the sample product. For each test sample, an even layer of product was applied to a substrate of roughened polymethyl methacrylate (PMMA) (Helioplate™ HD6 by HelioScreen, Creil, France). The substrate has a roughness of 5 μm to mimic skin topography. 32.5 mg of product was applied to the substrate at a concentration of 1.3 mg/cm². The product was allowed to air dry for at least 15 minutes, after which UV absorption curves were generated, and the underlying data analyzed, using the SPF-290S recording UV Spectrophotometer (Optometrics, Corp.). For each sample, four measurements were made and averaged together.

### Results

| | UVA/UVB | UVA1/UV | UVB | UVA | Critical | |
|---|---|---|---|---|---|---|
| | absor- | absor- | area | area | wave- | |
| | bance | bance | under | under | length | Absor- |
| Sample | ratio | ratio | curve | curve | (nm) | bance |
| Test | 0.795 | 0.86 | 37.84 | 30.08 | 377.1 | 0.744 |
| sample | | | | | | |
| (ZnO and | | | | | | |
| polymers) | | | | | | |
| Control 1 | 0.816 | 0.88 | 25.30 | 20.65 | 378.9 | 0.494 |
| (ZnO, no | | | | | | |
| polymers) | | | | | | |
| Control 2 | 0.420 | 0.66 | 1.21 | 0.52 | 381.0 | 0.032 |
| (polymers, | | | | | | |
| no ZnO) | | | | | | |

### Discussion of Results

The United States Food & Drug Administration requires that in order to claim “broad-spectrum” protection, the critical wavelength must be ≥370 nm. The in-vitro “critical wavelength” is the wavelength below which 90% of the area under the UV absorbance curve resides. Generally, the higher the critical wavelength, the greater the breadth of protection against the entire UV spectrum, 290 nm to 400 nm. All three samples met the critical wavelength requirement.

The FDA further stipulates that a broad-spectrum sunscreen product should exhibit a UVA1/UV absorbance ratio of ≥0.7. The test sample (zinc plus polymers) and control sample 1 (zinc only) meet these broad spectrum requirements. The UV absorption curves showed that the test sample and control sample 1 exhibited very little reduction in amplitude (absorbance) below 370 nm wavelength, followed by a sharp decline to 400 nm.

In contrast, control sample 2 (polymers, but no ZnO) exhibited a UVA1/UV ratio of 0.66. The UV absorption curve showed a pronounced and steady decline in amplitude (absorbance) from 290 nm to 370 nm, and remained low through 400 nm. This decline lowered the UVA1/UV ratio below 0.7, outside of the FDA requirement for broad spectrum protection.

SPF value is directly correlated to the area under the UVB portion of the absorbance curve (290-320 nm); the greater the area, the greater the SPF value. We note that for control sample 1 (conventional sunscreen product with zinc), the area under the UVB portion of the absorbance curve was 25.30. For control sample 2 (polymers but no zinc), the area under the UVB portion of the absorbance curve was only 1.21. Therefore, it was completely unexpected that the test sample (conventional sunscreen product with polymers added) would exhibit an area under the UVB portion of the absorbance curve of 37.84, far more than one would expect from an additive effect. In fact, the addition of acrylates/VA copolymer and acrylates copolymer to the ZnO-containing composition has synergistically boosted the area under the UV-B portion of the absorbance curve (and therefore synergistically boosted SPF) without adding more ZnO. Furthermore, although the control sample 2 (polymers, but no ZnO) did not meet the definition of “broad spectrum”, the test sample with ZnO and the two polymers did, due to the observed synergistic effect.

### Absorption Analysis 3

As noted above, compositions within the scope of the present invention may also be used as a primer, applied to the skin first, and a separate sunscreen composition applied on top. To demonstrate the effectiveness of this approach, a primer composition with no sunscreen agents was developed, comprising acrylates/VA copolymer and acrylates copolymer (see the table below). Next, a test sunscreen composition was selected, to be applied over the primer composition after the primer composition has dried on the substrate. For this, we selected Bobbie Brown™ Skin Long-Wear Weightless Foundation SPF15, which comprises 0.61% titanium dioxide (TiO₂) and chemical sunscreen octinoxate (3%). Following the procedure detailed above, we measured the area under the UV-B portion of the absorbance curve for the primer composition and the test sunscreen composition.

| Ingredient | Primer Composition |
|---|---|
| water | q.s. |
| acrylates/VA copolymer | 18.63 |
| HDI/PPG/polycaprolactone | 4.90 |
| crosspolymer | |
| alcohol | 3.00 |
| propanediol | 3.00 |
| acrylates copolymer | 1.00 |
| glycerine | 1.00 |
| phenoxyethanol | 0.89 |
| xanthan gum | 0.40 |
| silica | 0.10 |
| polyquaterium-6 | 0.06 |
| sodium dehydroacetate | 0.04 |
| disodium EDTA | 0.04 |
| sodium hyaluronate | 0.01 |
| laureth-20 | 0.01 |

### Results

| | | | UVB | UVA | |
|---|---|---|---|---|---|
| | UVA/UVB | UVA1/UV | area | area | Critical |
| | absorbance | absorbance | under | under | wavelength |
| Sample | ratio | ratio | curve | curve | (nm) |
| SPF 15 | 0.62 | 0.78 | 59.60 | 36.65 | 385.1 |
| product | | | | | |
| applied | | | | | |
| directly to | | | | | |
| PMMA plate | | | | | |
| Primer | 0.53 | 0.69 | 92.95 | 48.80 | 382.8 |
| applied first, | | | | | |
| SPF 15 | | | | | |
| product | | | | | |
| applied on | | | | | |
| top of primer | | | | | |

### Discussion of Results

For the commercially available SPF 15 product, the area under the UVB portion of the absorbance curve was 59.60 when applied directly to the PMMA plate. However, when the primer composition according to the present invention, which has very little sunscreen efficacy, was applied to the PMMA plate first, and then the SPF 15 product, the area under the UVB portion of the absorbance curve increased dramatically to 92.95, or a 56% increase. This translated to a boost in estimated SPF of slightly more than 50%.

We also note that the critical wavelength of the primer/sunscreen product combination has decreased slightly, but is still well above the 370 nm as required by the United States Food & Drug Administration for “broad-spectrum” claims. Thus, when used as a primer, a composition comprising specific combinations of acrylates/VA copolymer and acrylates copolymer are effective to boost the SPF of sunscreen products that comprise physical sunscreens.

### CONCLUDING REMARKS

It was not known at the outset that specific combinations of acrylates/VA copolymer and acrylates copolymer could be used to synergistically increases the effectiveness of compositions that comprise the physical sunscreen. It was further not clear that the incorporation of acrylates/VA copolymer and acrylates copolymer into a sunscreen product could be done in a commercially viable way to yield thermodynamically stable composition that feels comfortable on the skin. It was also not clear that specific combinations of acrylates/VA copolymer and acrylates copolymer could be used in a primer composition to boost the effectiveness of sunscreen compositions without increasing the level of physical sunscreen agents in the product.

What is claimed is:

1. A film forming composition comprising, by total weight of the composition: 5.0% to 30% of acrylates/VA copolymer;
acrylates copolymer, wherein: the ratio of acrylates/VA copolymer to acrylates copolymer is between 3.3:1 and 120:1;
20% to 70% of water;
1% to 20% of physical sunscreen agent, and
wherein, when the film forming composition is applied to a substrate and allowed to dry, the dried film will have an average porosity between 1 μm and 5 μm.

2. A composition according to claim 1 wherein the physical sunscreen agent is titanium dioxide, zinc oxide or a combination of the two.

3. A composition according to claim 1 wherein the ratio of acrylates/VA copolymer to acrylates copolymer is between 10:1 and 40:1.

4. A composition according to claim 3 wherein the ratio of acrylates/VA copolymer to acrylates copolymer is between 10:1 and 30:1.

5. A composition according to claim 4 wherein the ratio of acrylates/VA copolymer to acrylates copolymer is between 18:1 and 20:1.

6. A sunscreen treatment regimen comprising the steps of:
a. applying directly to the skin a film forming primer composition comprising, by total weight of the composition:
5.0% to 30% of acrylates/VA copolymer;
acrylates copolymer, wherein: the ratio of acrylates/VA copolymer to acrylates copolymer is between 3.3:1 and 120:1;
20% to 70% of water; and
no physical or chemical sunscreen agents;
wherein, when the primer composition is allowed to dry, the dried film has an average porosity between 1 μm and 5 μm;
b. allowing the primer composition to dry;
c. applying a sunscreen composition on top of the dried primer composition.

7. A composition according to claim 3 wherein the sunscreen composition comprises one or more physical sunscreen agents.
